(19) United States
(12) Patent Application Publication
(10) Pub. No.: US 2024/0317814 A1
(43) Pub. Date: Sep. 26, 2024

(54) Hybrid Recombinant Adeno-Associated Virus Serotype Between AAV9 and AAVrh74 with Reduced Liver Tropism

(71) Applicants: Genethon, Evry (FR); INSERM (Institut National de la Santé et de la Recherche Médicale), Paris (FR); Universite d'Evry Val d'Essonne, Evry (FR); Sorbonne Universite, Paris (FR); Association Institut de Myologie, Paris (FR)

(72) Inventors: Isabelle Richard, Corbeil (FR); Evelyne Gicquel, Vert-le-Petit (FR); Federico Mingozzi, Paris (FR)

(73) Assignees: Genethon, Evry (FR); INSERM (Institut National de la Santé et de la Recherche Médicale), Paris (FR); Universite d'Evry Val d'Essonne, Evry (FR); Sorbonne Universite, Paris (FR); Association Institut de Myologie, Paris (FR)

(21) Appl. No.: 18/735,636

(22) Filed: Jun. 6, 2024

Related U.S. Application Data
(63) Continuation of application No. 16/981,086, filed on Sep. 15, 2020, now Pat. No. 12,037,362.

(30) Foreign Application Priority Data
Apr. 5, 2018 (EP) 18305399.0

(51) Int. Cl. C07K 14/005; A61K 48/00; A61P 25/28; C07K 14/075; C12N 7/00; C12N 9/22; C12N 15/11; C12N 15/86

(52) U.S. Cl. CPC C07K 14/005; A61P 25/28; C07K 14/075; C12N 7/00; C12N 9/22; C12N 15/111; C12N 15/86; A61K 48/00; C12N 2310/20; C12N 2750/14121; C12N 2750/14122; C12N 2750/14143; C12N 2750/14152; C12N 2750/14171

(57) ABSTRACT

The invention relates to a recombinant adeno-associated virus (AAV) capsid protein, which is a hybrid between AAV serotype 9 (AAV9) and AAV serotype 74 (AAVrh74) capsid proteins, wherein said recombinant hybrid AAV capsid protein has a reduced liver tropism compared to the parent AAV9 and AAVrh74 capsid proteins. The invention relates also to the derived hybrid AAV serotype vector particles packaging a gene of interest and their use in gene therapy, in particular for treating neuromuscular genetic diseases.

### Sequence Listing Submission via Patent Center

The contents of the electronic sequence listing (sequencelisting.xml; Size: 65,063 bytes; and Date of Creation: Jun. 5, 2024) is herein incorporated by reference in its entirety.

### FIELD OF THE INVENTION

The present invention relates to a recombinant adeno-associated virus (AAV) capsid, which is a hybrid between AAV serotype 9 (AAV9) and AAV serotype rh74 (AAVrh74) capsid proteins having a reduced liver tropism compared to the parent AAV9 and AAVrh74 capsid proteins. The invention relates also to the derived hybrid AAV serotype vector particles packaging a gene of interest, and their use in gene therapy, in particular for treating neuromuscular genetic diseases.

### BACKGROUND OF THE INVENTION

Recombinant Adeno-Associated Virus (rAAV) vectors are widely used for in vivo gene transfer. rAAV vectors are non-enveloped vectors composed of a capsid of 20 nm of diameter and a single strand DNA of 4.7 kb. The genome carries two genes, rep and cap, flanked by two palindromic regions named Inverted terminal Repeats (ITR). The cap gene codes for three structural proteins VP1, VP2 and VP3 that compose the AAV capsid. VP1, VP2 and VP3 share the same C-terminal end which is all of VP3. Using AAV2 has a reference, VP1 has a 735 amino acid sequence (GenBank YP_680426); VP2 (598 amino acids) starts at the Threonine 138 (T138) and VP3 (533 amino acids) starts at the methionine 203 (M203). AAV serotypes are defined by their capsid. Different serotypes exist, each of them displaying its own tissue targeting specificity. Therefore, the choice of using a serotype depends on the tissue to transduce. Skeletal muscle and liver tissues are infected and transduced efficiently by different serotypes of AAV vectors such as AAV8, AAV9 and AAV-rh74. Chimeric or hybrid AAV serotypes have been generated by exchanging fragments of capsid sequences between capsids of different naturally occurring AAV serotypes, in order to increase AAV transduction efficiency or increase AAV tropism to a cell or tissue type of interest.

Hybrid AAV capsids were generated by combining structural domains of capsids of AAV8 and AAV serotypes isolated from primate brain. The resulting AAV hybrid serotypes can transduce retinal tissue in human and mice with no increase in efficiency compared to AAV2 and AAV5 vectors (Charbel Issa et al., PLOS ONE, 2013, 8, e60361). However, one of the hybrid AAV serotype shows improved transduction efficiency for fat tissue compared to AAV1, AAV8 and AAV9 (Liu et al., Molecular Therapy, 2014, 1, 8, doi:10.1038/mtm). WO 2015/191508 discloses recombinant hybrid AAV capsids generated by exchanging variable regions of AAV capsids from various species (human, primate, avian, snake, bovine.), in particular AAV capsids with central nervous system tropism to generate CNS specific chimeric capsids.

WO 2017/096164 discloses recombinant hybrid AAV capsids between AAV1, AAV2, AAV3b, AAV6 and AAV8 serotypes exhibiting enhanced human skeletal muscle tropism. However, all naturally occurring AAV serotypes and variants tested to date have a propensity to accumulate within the liver. This causes problems, in particular when the AAV vector is administered by the systemic route. Firstly, a transgene aimed to be expressed in muscle may have toxic effects on the liver. Secondly, AAV vector entry in liver reduces the amount of vector available for skeletal muscles. Consequently, higher doses of AAV vectors are required. This increases liver toxicity and cost of vector production.

Tissue-specific promoters and microRNA-based gene regulation strategies have been used to segregate gene expression patterns among different tissue types. However, such regulatory strategies do not preclude sequestration of AAV vector genomes in off-target organs such as the liver after systemic administration.

Attenuation of heparin binding by mutating the basic residues R585 or R588 of the capsid protein was shown to abolish heparin sulfate binding and reduce the liver tropism of AAV2-derived vectors (Asokan et al., Nat. Biotechnol., 2010, 28, 79-82). However, this strategy can only work for serotypes like AAV2 and AAV6 whose liver tropism is determined by basic residues binding to heparin.

Therefore, there is a need for new AAV vectors, having a liver tropism which is much lower than their muscle tropism. In addition, new vectors that could infect muscles efficiently but could not infect the liver nor the brain would be even more desirable.

### SUMMARY OF THE INVENTION

The inventors have generated new hybrid AAV serotypes using a combination of two serotypes that infect efficiently the muscle and liver tissues, AAV9 and AAV-rh74. Two new hybrid AAV serotypes were generated using the swapping of a variable region of the cap gene between the AAV9 and AAVrh74 serotypes (FIGS. 1A and 1B). Surprisingly, the liver tropism of the parent AAV9 and AAVrh74 was lost in the hybrid AAV serotype (FIGS. 4C and 4D). At the same time, the hybrid AAV serotype exhibited high titer AAV vector production and high level gene transduction efficiencies in skeletal and cardiac muscle tissues.

The new hybrid AAV serotypes are useful in gene therapy of neuromuscular disorders, including genetic diseases, autoimmune diseases, neurodegenerative diseases and cancer.

Therefore, the invention encompasses a hybrid recombinant AAV capsid between AAV9 and AAVrh74 capsids with reduced liver tropism, AAV vector particles comprising the hybrid recombinant AAV capsid, compositions comprising the hybrid AAV serotype vector particles, and methods of making and using said hybrid AAV serotype vector particles and compositions, in particular in gene therapy.

### DETAILED DESCRIPTION OF THE INVENTION

### Recombinant Hybrid AAV Capsid Protein

One aspect of the invention relates to a recombinant adeno-associated virus (AAV) capsid protein, which is a hybrid between AAV serotype 9 (AAV9) and AAV serotype 74 (AAVrh74) capsid proteins, wherein said recombinant hybrid AAV capsid protein has reduced liver tropism compared to its parent AAV9 and AAVrh74 capsid proteins.

As used herein, the term “tropism” refers to the specificity of an AAV capsid protein present in an AAV viral particle, for infecting a particular type of cell or tissue.

The tropism of an AAV capsid for a particular type of cell or tissue may be determined by measuring the ability of AAV vector particles comprising the hybrid AAV capsid protein to infect or to transduce a particular type of cell or tissue, using standard assays that are well-known in the art such as those disclosed in the examples of the present application.

As used herein, the term “liver tropism” or “hepatic tropism” refers to the tropism for liver or hepatic tissue and cells, including hepatocytes.

According to the invention, the liver tropism of the hybrid AAV capsid protein is reduced by at least 20%, 30%, 40%, 50% or more; preferably at least 50%, 60% 70%, 80%, 90% or 99% compared to the liver tropism of the parent AAV9 or AAVrh74 capsid protein.

According to the invention, the hybrid AAV capsid protein has tropism for muscle cells and tissues.

Muscle tissues include in particular cardiac and skeletal muscle tissues.

As used herein, the term “muscle cells” refers to myocytes, myotubes, myoblasts, and/or satellite cells.

In some embodiments, the muscle tropism of the hybrid AAV capsid protein is similar to that of its parent AAV9 and/or AAVrh74 capsid proteins. Preferably, the muscle tropism of the hybrid AAV capsid protein is equivalent to at least 50%, 60%, 70%, 80%, 90%, 99% or more of that of the parent AAV9 and/or AAVrh74 capsid protein.

In some embodiments, the hybrid AAV capsid protein is a hybrid VP1, VP2 or VP3 protein.

In some embodiments, the hybrid AAV capsid protein has tropism for at least skeletal muscle tissue. In some preferred embodiments, the hybrid AAV capsid protein has tropism for both skeletal and cardiac muscle tissues. An example of this type of hybrid is the hybrid AAV capsid of SEQ ID NO: 3 (named Hybrid Cap9-rh74 in the examples). This type of hybrid AAV capsid is useful for the treatment of cardiac and skeletal muscle disorders.

The hybrid AAV capsid protein according to the invention may be derived from any AAV9 and AAVrh74 capsid protein sequences; such sequences are well-known in the art and available in public sequence data base. For example, AAV9 capsid protein corresponds to GenBank accession numbers: AY530579.1; SEQ ID NO: 123 of WO 2005/033321; SEQ ID NO: 1 of WO 2012/112832; AAV9 capsid variants in which one or more of the native residues at positions 271 (D), 446 (Y), and 470 (N) are replaced with another amino acid, preferably alanine as disclosed in WO 2012/112832; AAV9 capsid variants at one or more of positions K143R, T251A, S499A, S669A and S490A as disclosed in US 2014/0162319. AAVrh74 capsid protein corresponds to SEQ ID NO: 1 of WO 2015/013313; SEQ ID NO: 6 of WO 2006/110689; SEQ ID NO: 1 of WO 2013/123503; SEQ ID NO: 4 of WO 2013/158879; and K137R, K333R, K550R, K552R, K569R, K691R, K695R, K709R variants and combination thereof.

In some embodiments, the hybrid AAV capsid protein according to the invention is derived from the AAV9 capsid protein of SEQ ID NO: 1 (GenBank AY530579.1) and the AAVrh74 protein of SEQ ID NO: 2.

In some embodiments, the hybrid AAV capsid protein according to the invention results from the replacement of a variable region in the AAV9 or AAVrh74 capsid sequence with the corresponding variable region of the other AAV serotype capsid sequence,
wherein the variable region of AAV9 capsid corresponds to the sequence situated from any one of positions 331 to 493 to any one of positions 556 to 736 in AAV9 capsid of SEQ ID NO: 1 (reference sequence), or a fragment of at least 10, 15, 20, 25, 30, 35, 40, 45, 50, 55 or 60 consecutive amino acids of the sequence situated from positions 493 to 556 in AAV9 capsid of SEQ ID NO: 1, and
the variable region of AAVrh74 capsid corresponds to the sequence situated from any one of positions 332 to 495 to any one of positions 558 to 738 in AAVrh74 capsid of SEQ ID NO: 2 (reference sequence), or a fragment of at least 10, 15, 20, 25, 30, 35, 40, 45, 50, 55 or 60 consecutive amino acids of the sequence situated from positions 495 to 558 in AAVrh74 capsid of SEQ ID NO: 2.

The invention encompasses hybrid AAV capsid proteins derived from any AAV9 and AAVrh74 capsid protein sequences by replacement of a variable region region in the AAV9 or AAVrh74 capsid sequence with the corresponding variable region of the other AAV serotype capsid sequence, as defined above. According to the invention, the variable region is defined using AAV9 capsid of SEQ ID NO: 1 and AAVrh74 capsid of SEQ ID NO: 2 as reference. After sequence alignment of any other AAV9 capsid sequence with SEQ ID NO: 1 or any of other AAVrh74 capsid sequence with SEQ ID NO: 2, using standard protein sequence alignment programs that are well-known in the art, such as for example BLAST, FASTA, CLUSTALW, and the like, a person skilled in the art can easily obtained the corresponding positions of the variable region in other AAV9 or AAVrh74 capsid sequences.

In some preferred embodiments, the hybrid AAV capsid protein according to the invention results from the replacement of the variable region corresponding to that situated from positions 449 to 609 in the AAV9 capsid sequence of SEQ ID NO: 1 or from positions 450 to 611 in the AAVrh74 capsid sequence of SEQ ID NO: 2 with the corresponding variable region of the other serotype.

In some embodiments, said hybrid AAV capsid protein comprises a sequence selected from the group consisting of the sequences SEQ ID NO: 3 and SEQ ID NO: 4, the sequences having at least 85%, 90%, 95%, 97%, 98% or 99% identity with said sequences, and the fragment thereof corresponding to VP2 or VP3 capsid protein. VP2 corresponds to the amino acid sequence from T138 to the end of SEQ ID NO: 3 or 4. VP3 corresponds to the amino acid sequence from M203 to the end of SEQ ID NO: 3 or from M204 to the end of SEQ ID NO: 4.

SEQ ID NO: 3 is derived from AAV9 capsid protein of SEQ ID NO: 1 by replacement of AAV9 variable region (positions 449 to 609 of SEQ ID NO: 1) with the variable region of AAVrh74 capsid protein (positions 450 to 611 of SEQ ID NO: 2); the corresponding hybrid is named Hybrid Cap9-rh74 in the examples. VP2 corresponds to the amino acid sequence from T138 to the end of SEQ ID NO: 3. VP3 corresponds to the amino acid sequence from M203 to the end of SEQ ID NO: 3.

SEQ ID NO: 4 is derived from AAVrh74 capsid protein of SEQ ID NO: 2 by replacement of rh74 variable region (positions 450 to 611 of SEQ ID NO: 2) with the variable region of AAV9 capsid protein (positions 449 to 609 of SEQ ID NO: 1); the corresponding hybrid is named Hybrid Caprh74-9 in the examples. VP2 corresponds to the amino acid sequence from T138 to the end of SEQ ID NO: 4. VP3 corresponds to the amino acid sequence from M204 to the end of SEQ ID NO: 4.

In some preferred embodiments, the hybrid AAV capsid protein according to the invention is derived from AAV9 capsid protein by replacement of a variable region of AAV9 capsid sequence with the corresponding variable region of AAVrh74 capsid sequence as defined above, preferably the hybrid AAV capsid protein comprises the replacement of the variable region corresponding to that situated from positions 449 to 609 in AAV9 capsid of SEQ ID NO: 1 with the variable region corresponding to that situated from positions 450 to 611 in AAVrh74 capsid of SEQ ID NO: 2. Preferably, said hybrid AAV capsid protein comprises a sequence selected from the group consisting of the sequence of SEQ ID NO: 3 and the sequences having at least 85%, 90%, 95%, 97%, 98% or 99% identity with said sequence; more preferably which comprises the sequence of SEQ ID NO: 3.

The term “identity” refers to the sequence similarity between two polypeptide molecules or between two nucleic acid molecules. When a position in both compared sequences is occupied by the same base or same amino acid residue, then the respective molecules are identical at that position. The percentage of identity between two sequences corresponds to the number of matching positions shared by the two sequences divided by the number of positions compared and multiplied by 100. Generally, a comparison is made when two sequences are aligned to give maximum identity. The identity may be calculated by alignment using, for example, the GCG (Genetics Computer Group, Program Manual for the GCG Package, Version 7, Madison, Wisconsin) pileup program, or any of sequence comparison algorithms such as BLAST, FASTA or CLUSTALW.

In some embodiments, the hybrid AAV capsid protein of the invention generates high yields of recombinant AAV vector particles. Preferably, the titer of the hybrid capsid recombinant AAV vector is equal or superior to 1011 viral genomes per mL (vg/mL). High yields of recombinant AAV vector particles are useful for gene therapy applications.

In some embodiments, the hybrid AAV capsid protein of the invention further comprises, additional modifications, for example modifications which increase the targeting of skeletal or cardiac muscle tissue by AAV vectors. A non-limiting example is the fusion of Anthopleurin-B to the N-terminus of AAV VP2 capsid protein (Finet et al., Virology, 2018, 513, 43-51). Another modification is the insertion of a peptide into a site exposed on the capsid surface, in particular around position 588 according to the numbering in SEQ ID NO: 1. Non-limiting examples of such peptides are disclosed in Michelfelder et al. (PLOS ONE, 2009, 4, e5122). The insertion site is advantageously from positions 587 to 592 according to the numbering in SEQ ID NO: 1. The insertion of the peptide may or may not cause the deletion of some or all of the residue(s) from the insertion site. The peptide has advantageously a sequence of no more than 20 amino acids which may include fixed sequences of no more than five amino acids at its N-and/or C-terminal ends, such as for example GQSG (SEQ ID NO: 35) and AQAA (SEQ ID NO: 36), respectively at the N-and C-terminal end of the peptide.

In some embodiments, the peptide comprises or consists of a sequence selected from the group consisting of SEQ ID NO: 12 to 34. Preferably, said peptide is flanked by GQSG (SEQ ID NO: 35) and AQAA (SEQ ID NO: 36), respectively at its N-and C-terminal end. The peptide advantageously replaces all the residues from positions 587 to 592 of the AAV capsid protein according to the numbering in SEQ ID NO: 1. The peptide advantageously increases the targeting of cardiac muscle tissue and eventually also of skeletal muscle tissue. In some preferred embodiment, the peptide-modified hybrid AAV capsid protein comprises or consists of a sequence selected from the group consisting of SEQ ID NO: 9 and the sequences having at least 85%, 90%, 95%, 97%, 98% or 99% identity with said sequence; more preferably which comprises the sequence of SEQ ID NO:9. SEQ ID NO: 9 is derived from the hybrid Cap9-rh74 of SEQ ID NO: 3 by the insertion of the peptide of SEQ ID NO: 12. The invention encompasses also AAV VP1 and VP2 chimeric capsid proteins derived from the AAV9/rh74 hybrid VP3 capsid protein according to the invention, wherein the VP1-specific N-terminal region and/or VP2-specific N-terminal region are from a natural or artificial AAV serotype other than AAV9 and AAVrh74.

In some embodiments, the AAV VP1 chimeric capsid protein comprises:
(i) a VP1-specific N-terminal region having a sequence from natural or artificial AAV serotype other than AAV9 and AAVrh74,
(ii) a VP2-specific N-terminal region having a sequence from AAV9, AAVrh74 or natural or artificial AAV serotype other than AAV9 and AAVrh74, and
(iii) a VP3 C-terminal region having the sequence of a hybrid VP3 protein according to the invention.
In some embodiments, the AAV VP2 chimeric capsid proteins comprises
(i) a VP2-specific N-terminal region having a sequence from natural or artificial AAV serotype other than AAV9 and AAVrh74, and
(ii) a VP3 C-terminal region having the sequence of a hybrid VP3 protein according to the invention.

### Polynucleotide, Vector, and Use for AAV Vector Production

Another aspect of the invention is a polynucleotide encoding the recombinant hybrid AAV capsid protein in expressible form. The polynucleotide may be DNA, RNA or a synthetic or semi-synthetic nucleic acid.

In some embodiments, the polynucleotide is a AAV9/rh74 hybrid cap gene encoding hybrid VP1, VP2 and VP3 capsid proteins according to the invention. In some preferred embodiments, the polynucleotide comprises the sequence SEQ ID NO: 5 (encoding the hybrid AAV capsid protein of SEQ ID NO: 3) or the sequence SEQ ID NO: 7 (encoding the hybrid AAV capsid protein of SEQ ID NO: 4).

In some other embodiments, the polynucleotide is a chimeric cap gene which codes for a AAV9/rh74 hybrid VP3 capsid protein according to the invention and a chimeric VP1 capsid protein, and maybe also a chimeric VP2 capsid protein wherein the VP1-specific N-terminal region, and maybe also the VP2-specific N-terminal region, are from a natural or artificial AAV serotype other than AAV9 and AAVrh74. Such chimeric cap gene may be generated by any suitable technique, using the coding sequence for an AAV9/rh74 hybrid VP3 capsid protein according to the invention in combination with heterologous sequences which may be obtained from different selected AAV serotypes, non-contiguous portions of the same AAV serotypes, from a non-viral AAV source or from a non-viral source.

In some embodiments, the polynucleotide further encodes AAV Replicase (Rep) protein in expressible form, preferably Rep from AAV2.

The polynucleotide is advantageously inserted into a recombinant vector, which includes, in a non-limiting manner, linear or circular DNA or RNA molecules consisting of chromosomal, non-chromosomal, synthetic or semi-synthetic nucleic acids, such as in particular viral vectors, plasmid or RNA vectors.

Numerous vectors into which a nucleic acid molecule of interest can be inserted in order to introduce it into and maintain it in a eukaryotic host cell are known per se; the choice of an appropriate vector depends on the use envisioned for this vector (for example, replication of the sequence of interest, expression of this sequence, maintaining of this sequence in extrachromosomal form, or else integration into the chromosomal material of the host), and also on the nature of the host cell.

In some embodiments, the vector is a plasmid.

The recombinant vector for use in the present invention is an expression vector comprising appropriate means for expression of the hybrid AAV capsid protein, and maybe also AAV Rep protein. Usually, each coding sequence (hybrid AAV Cap and AAV Rep) is inserted in a separate expression cassette either in the same vector or separately. Each expression cassette comprises the coding sequence (open reading frame or ORF) functionally linked to the regulatory sequences which allow the expression of the corresponding protein in AAV producer cells, such as in particular promoter, promoter/enhancer, initiation codon (ATG), stop codon, transcription termination signal. Alternatively, the hybrid AAV Cap and the AAV Rep proteins may be expressed from a unique expression cassette using an Internal Ribosome Entry Site (IRES) inserted between the two coding sequences or a viral 2A peptide. In addition, the codon sequences encoding the hybrid AAV Cap, and AAV Rep if present, are advantageously optimized for expression in AAV producer cells, in particular human producer cells.

The vector, preferably a recombinant plasmid, is useful for producing hybrid AAV vectors comprising the hybrid AAV capsid protein of the invention, using standard AAV production methods that are well-known in the art (Review in Aponte-Ubillus et al., Applied Microbiology and Biotechnology, 2018, 102: 1045-1054).

Following co-transfection, the cells are incubated for a time sufficient to allow the production of AAV vector particles, the cells are then harvested, lysed, and AAV vector particles are purified by standard purification methods such as for example Cesium Chloride density gradient ultracentrifugation.

### AAV Particle, Pharmaceutical Composition and Therapeutic Uses

Another aspect of the invention is an AAV particle comprising the hybrid recombinant AAV capsid protein of the invention. The AAV particle may comprise hybrid VP1, VP2 and VP3 capsid proteins encoded by a hybrid cap gene according to the invention. Alternatively or additionally, the AAV particle may comprise chimeric VP1 and VP2 capsid proteins and a hybrid VP3 protein encoded by a chimeric cap gene according to the invention.

In some embodiments, the AAV particle is a mosaic AAV particle further comprising another AAV capsid protein from a natural or artificial AAV serotype other than AAV9 and AAVrh74 serotype, wherein the mosaic AAV particle has a reduced liver tropism compared to AAV9 and AAVrh74 serotypes. An artificial AAV serotype may be with no limitation, a chimeric AAV capsid, a recombinant AAV capsid, or a humanized AAV capsid. Such an artificial capsid may be generated by any suitable technique, using a selected AAV sequence (e.g. a fragment of a VP1 capsid protein) in combination with heterologous sequences which may be obtained from a different selected AAV serotype, non-contiguous portions of the same AAV serotype, from a non-viral AAV source or from a non-viral source.

Preferably, the AAV particle is an AAV vector particle. The genome of the AAV vector may either be a single-stranded or self-complementary double-stranded genome (McCarty et al, Gene Therapy, 2003 December, 10 (26), 2112-2118). Self-complementary vectors are generated by deleting the terminal resolution site (trs) from one of the AAV terminal repeats. These modified vectors, whose replicating genome is half the length of the wild-type AAV genome have the tendency to package DNA dimers. The AAV genome is flanked by ITRs. In particular embodiments, the AAV vector is a pseudotyped vector, i.e. its genome and capsid are derived from AAVs of different serotypes. In some preferred embodiments, the genome of the pseudotyped vector is derived from AAV2.

In some preferred embodiments, the AAV vector particle is packaging a gene of interest.

The AAV particle may be obtained using the method of producing recombinant AAV vector particles of the invention.

By “gene of interest”, it is meant a gene useful for a particular application, such as with no limitation, diagnosis, reporting, modifying, therapy and genome editing.

For example, the gene of interest may be a therapeutic gene, a reporter gene or a genome-editing enzyme.

By “gene of interest for therapy”, “gene of therapeutic interest”, or “heterologous gene of interest”, it is meant a therapeutic gene or a gene encoding a therapeutic protein, peptide or RNA.

The gene of interest is any nucleic acid sequence capable of modifying a target gene or target cellular pathway, in particular in muscle cells. For example, the gene may modify the expression, sequence or regulation of the target gene or cellular pathway. In some embodiments, the gene of interest is a functional version of a gene or a fragment thereof. The functional version of said gene includes the wild-type gene, a variant gene such as variants belonging to the same family and others, or a truncated version, which preserves the functionality of the encoded protein at least partially. A functional version of a gene is useful for replacement or additive gene therapy to replace a gene, which is deficient or non-functional in a patient. In other embodiments, the gene of interest is a gene which inactivates a dominant allele causing an autosomal dominant genetic disease. A fragment of a gene is useful as recombination template for use in combination with a genome editing enzyme.

Alternatively, the gene of interest may encode a protein of interest for a particular application, (for example an antibody or antibody fragment, a genome-editing enzyme) or a RNA. In some embodiments, the protein is a therapeutic protein including a therapeutic antibody or antibody fragment, or a genome-editing enzyme. In some embodiments, the RNA is a therapeutic RNA. The gene of interest is a functional gene able to produce the encoded protein, peptide or RNA in the target cells of the disease, in particular muscle cells. The AAV viral vector comprises the gene of interest in a form expressible in muscle cells, including cardiac and skeletal muscle cells. In particular, the gene of interest is operatively linked to a ubiquitous, tissue-specific or inducible promoter which is functional in muscle cells. The gene of interest may be inserted in an expression cassette further comprising polyA sequences.

The RNA is advantageously complementary to a target DNA or RNA sequence or binds to a target protein. For example, the RNA is an interfering RNA such as a shRNA, a microRNA, a guide RNA (gRNA) for use in combination with a Cas enzyme or similar enzyme for genome editing, an antisense RNA capable of exon skipping such as a modified small nuclear RNA (snRNA) or a long non-coding RNA. The interfering RNA or microRNA may be used to regulate the expression of a target gene involved in muscle disease. The guide RNA in complex with a Cas enzyme or similar enzyme for genome editing may be used to modify the sequence of a target gene, in particular to correct the sequence of a mutated/deficient gene or to modify the expression of a target gene involved in a disease, in particular a neuromuscular disease. The antisense RNA capable of exon skipping is used in particular to correct a reading frame and restore expression of a deficient gene having a disrupted reading frame. In some embodiments, the RNA is a therapeutic RNA.

The genome-editing enzyme according to the invention is any enzyme or enzyme complex capable of modifying a target gene or target cellular pathway, in particular in muscle cells. For example, the genome-editing enzyme may modify the expression, sequence or regulation of the target gene or cellular pathway. The genome-editing enzyme is advantageously an engineered nuclease, such as with no limitations, a meganuclease, zinc finger nuclease (ZFN), transcription activator-like effector-based nuclease (TALENs), Cas enzyme from clustered regularly interspaced palindromic repeats (CRISPR)-Cas system and similar enzymes. The genome-editing enzyme, in particular an engineered nuclease such as Cas enzyme and similar enzymes, may be a functional nuclease which generates a double-strand break (DSB) in the target genomic locus and is used for site-specific genome editing applications, including with no limitations: gene correction, gene replacement, gene knock-in, gene knock-out, mutagenesis, chromosome translocation, chromosome deletion, and the like. For site-specific genome editing applications, the genome-editing enzyme, in particular an engineered nuclease such as Cas enzyme and similar enzymes may be used in combination with a homologous recombination (HR) matrix or template (also named DNA donor template) which modifies the target genomic locus by double-strand break (DSB)-induced homologous recombination. In particular, the HR template may introduce a transgene of interest into the target genomic locus or repair a mutation in the target genomic locus, preferably in an abnormal or deficient gene causing a neuromuscular disease. Alternatively, the genome-editing enzyme, such as Cas enzyme and similar enzymes may be engineered to become nuclease-deficient and used as DNA-binding protein for various genome engineering applications such as with no limitation: transcriptional activation, transcriptional repression, epigenome modification, genome imaging, DNA or RNA pull-down and the like.

Another aspect of the invention is a pharmaceutical composition comprising a therapeutically effective amount of AAV particles comprising the hybrid recombinant AAV capsid protein of the invention, preferably AAV vector particles packaging a therapeutic gene of interest.

In some embodiments of the invention, the pharmaceutical composition of the invention is for use as a medicament, in particular in gene therapy. The invention encompasses the use of the pharmaceutical composition of the invention as a medicament, in particular for the treatment of a disease by gene therapy.

Gene therapy can be performed by gene transfer, gene editing, exon skipping, RNA-interference, trans-splicing or any other genetic modification of any coding or regulatory sequences in the cell, including those included in the nucleus, mitochondria or as commensal nucleic acid such as with no limitation viral sequences contained in cells.

The two main types of gene therapy are the following:
a therapy aiming to provide a functional replacement gene for a deficient/abnormal gene: this is replacement or additive gene therapy;
a therapy aiming at gene or genome editing: in such a case, the purpose is to provide to a cell the necessary tools to correct the sequence or modify the expression or regulation of a deficient/abnormal gene so that a functional gene is expressed or an abnormal gene is suppressed (inactivated): this is gene editing therapy.

In additive gene therapy, the gene of interest may be a functional version of a gene, which is deficient or mutated in a patient, as is the case for example in a genetic disease. In such a case, the gene of interest will restore the expression of a functional gene.

Gene or genome editing uses one or more gene(s) of interest, such as:
(i) a gene encoding a therapeutic RNA as defined above such as an interfering RNA like a shRNA or a microRNA, a guide RNA (gRNA) for use in combination with a Cas enzyme or similar enzyme, or an antisense RNA capable of exon skipping such as a modified small nuclear RNA (snRNA); and
(ii) a gene encoding a genome-editing enzyme as defined above such as an engineered nuclease like a meganuclease, zinc finger nuclease (ZFN), transcription activator-like effector-based nuclease (TALENs), Cas enzyme or similar enzymes; or a combination of such genes, and maybe also a fragment of a functional version of a gene for use as recombination template, as defined above.

Gene therapy is used for treating various diseases, including with no limitations, genetic diseases, in particular neuromuscular genetic disorders, cancer, neurodegenerative diseases and auto-immune diseases.

In some embodiments, gene therapy is used for treating diseases affecting muscle tissues, in particular skeletal muscle tissue and/or cardiac tissue, such as with no-limitations: neuromuscular genetic disorders, cardiomyopathies, rhabdomyosarcomas, Polymyositis, Dermatomyositis, juvenile polymyositis and others.

Examples of mutated genes in neuromuscular genetic disorders that can be targeted by gene therapy using the pharmaceutical composition of the invention are listed in the following tables:

### Muscular Dystrophies

| Gene | Protein |
|---|---|
| DMD | Dystrophin |
| EMD | Emerin |
| FHL1 | Four and a half LIM domain 1 |
| LMNA | Lamin A/C |
| SYNE1 | Spectrin repeat containing, nuclear envelope 1 (nesprin 1) |
| SYNE2 | Spectrin repeat containing, nuclear envelope 2 (nesprin 2) |
| TMEM43 | Transmembrane protein 43 |
| TOR1AIP1 | Torsin A interacting protein 1 |
| DUX4 | Double homeobox 4 |
| SMCHD1 | Structural maintenance of chromosomes flexible hinge |
| | domain containing 1 |
| PTRF | Polymerase I and transcript release factor |
| MYOT | Myotilin |
| CAV3 | Caveolin 3 |
| DNAJB6 | HSP-40 homologue, subfamily B. number 6 |
| DES | Desmin |
| TNPO3 | Transportin 3 |
| HNRNPDL | Heterogeneous nuclear ribonucleoprotein D-like |
| CAPN3 | Calpain 3 |
| DYSF | Dysferlin |
| SGCG | Gamma sarcoglycan |
| SGCA | Alpha sarcoglycan |
| SGCB | Beta sarcoglycan |
| SGCD | Delta-sarcoglycan |
| TCAP | Telethonin |
| TRIM32 | Tripartite motif-containing 32 |
| FKRP | Fukutin-related protein |
| TTN | Titin |
| POMT1 | Protein-O-mannosyltransferase 1 |
| ANO5 | Anoctamin 5 |
| FKTN | Fukutin |
| POMT2 | Protein-O-mannosyltransferase 2 |
| POMGNT1 | O-linked mannose beta1,2-N-acetylglucosaminyltransferase |
| PLEC | Plectin |
| TRAPPC11 | trafficking protein particle complex 11 |
| GMPPB | GDP-mannose pyrophosphorylase B |
| DAG1 | Dystroglycan1 |
| DPM3 | Dolichyl-phosphate mannosyltransferase polypeptide 3 |
| ISPD | Isoprenoid synthase domain containing |
| VCP | Valosin-containing protein |
| LIMS2 | LIM and senescent cell antigen-like domains 2 |
| GAA | Glucosidase alpha, acid |

### Congenital Muscular Dystrophies

| Gene | Protein |
|---|---|
| LAMA2 | Laminin alpha 2 chain of merosin |
| COL6A1 | Alpha 1 type VI collagen |
| COL6A2 | Alpha 2 type VI collagen |
| COL6A3 | Alpha 3 type VI collagen |
| SEPN1 | Selenoprotein N1 |
| FHL1 | Four and a half LIM domain 1 |
| ITGA7 | Integrin alpha 7 precursor |
| DNM2 | Dynamin 2 |
| TCAP | Telethonin |
| LMNA | Lamin A/C |
| FKTN | Fukutin |
| POMT1 | Protein-O-mannosyltransferase 1 |
| POMT2 | Protein-O-mannosyltransferase 2 |
| FKRP | Fukutin-related protein |
| POMGNT1 | O-linked mannose beta1,2-N-acetylglucosaminyltransferase |
| ISPD | Isoprenoid synthase domain containing |
| POMGNT2 | protein O-linked mannose N-acetylglucosaminyltransferase |
| | 2 |
| B3GNT1 | UDP-GlcNAc:betaGal beta-1,3-N-acetylglucosaminyl- |
| | transferase 1 |
| GMPPB | GDP-mannose pyrophosphorylase B |
| LARGE | Like-glycosyltransferase |
| DPM1 | Dolichyl-phosphate mannosyltransferase 1, catalytic subunit |
| DPM2 | Dolichyl-phosphate mannosyltransferase polypeptide 2, |
| | regulatory subunit |
| ALG13 | UDP-N-acetylglucosami-nyltransferase |
| B3GALNT2 | Beta-1,3-N-acetylgalacto-saminyltransferase 2 |
| TMEM5 | Transmembrane protein 5 |
| POMK | Protein-O-mannose kinase |
| CHKB | Choline kinase beta |
| ACTA1 | Alpha actin, skeletal muscle |
| TRAPPC11 | trafficking protein particle complex 11 |

### Congenital Myopathies

| Gene | Protein |
|---|---|
| TPM3 | Tropomyosin 3 |
| NEB | Nebulin |
| ACTA1 | Alpha actin, skeletal muscle |
| TPM2 | Tropomyosin 2 (beta) |
| TNNT1 | Slow troponin T |
| KBTBD13 | Kelch repeat and BTB (POZ) domain containing 13 |
| CFL2 | Cofilin 2 (muscle) |
| KLHL40 | Kelch-like family member 40 |
| KLHL41 | Kelch-like family member 41 |
| LMOD3 | Leiomodin 3 (fetal) |
| SEPN1 | Selenoprotein N1 |
| RYR1 | Ryanodine receptor 1 (skeletal) |
| MYH7 | Myosin, heavy polypeptide 7, cardiac muscle, beta |
| MTM1 | Myotubularin |
| DNM2 | Dynamin 2 |
| BIN1 | Amphiphysin |
| TTN | Titin |
| SPEG | SPEG complex locus |
| MEGF10 | Multiple EGF-like-domains 10 |
| MYH2 | Myosin, heavy polypeptide 2, skeletal muscle |
| MYBPC3 | Cardiac myosin binding protein-C |
| CNTN1 | Contactin-1 |
| TRIM32 | Tripartite motif-containing 32 |
| PTPLA | Protein tyrosine phosphatase-like (3-Hydroxyacyl-CoA |
| | dehydratase |
| CACNA1S | Calcium channel, voltage-dependent, L type, alpha 1S |
| | subunit |

### Distal Myopathies

| Gene | |
|---|---|
| symbol | protein |
| DYSF | Dysferlin |
| TTN | Titin |
| GNE | UDP-N-acetylglucosamine-2- epimerase/N-acetylmannosamine |
| | kinase |
| MYH7 | Myosin, heavy polypeptide 7, cardiac muscle, beta |
| MATR3 | Matrin 3 |
| TIA1 | Cytotoxic granuleassociated RNA binding protein |
| MYOT | Myotilin |
| NEB | Nebulin |
| CAV3 | Caveolin 3 |
| LDB3 | LIM domain binding 3 |
| ANO5 | Anoctamin 5 |
| DNM2 | Dynamin 2 |
| KLHL9 | Kelch-like homologue 9 |
| FLNC | Filamin C, gamma (actin-binding protein - 280) |
| VCP | Valosin-containing protein |

### Other Myopathies

| Gene | |
|---|---|
| symbol | protein |
| ISCU | Iron-sulfur cluster scaffold homolog (E. coli) |
| MSTN | Myostatin |
| FHL1 | Four and a half LIM domain 1 |
| BAG3 | BCL2-associated athanogene 3 |
| ACVR1 | Activin A receptor, type II-like kinase 2 |
| MYOT | Myotilin |
| FLNC | Filamin C, gamma (actin-binding protein - 280) |
| LDB3 | LIM domain binding 3 |
| LAMP2 | Lysosomal-associated membrane protein 2 precursor |
| VCP | Valosin-containing protein |
| CAV3 | Caveolin 3 |
| SEPN1 | Selenoprotein N1 |
| CRYAB | Crystallin, alpha B |
| DES | Desmin |
| VMA21 | VMA21 Vacuolar H+-ATPase Homolog (S. Cerevisiae) |
| PLEC | plectin |
| PABPN1 | Poly(A) binding protein, nuclear 1 |
| TTN | Titin |
| RYR1 | Ryanodine receptor 1 (skeletal) |
| CLN3 | Ceroid-lipofuscinosis, neuronal 3 (=battenin) |
| TRIM54 | |
| TRIM63 | Tripartite motif containing 63, E3 ubiquitin protein ligase |

### Myotonic Syndromes

| Gene | protein |
|---|---|
| DMPK | Myotonic dystrophy protein kinase |
| CNPB | Cellular nucleic acid-binding protein |
| CLCN1 | Chloride channel 1, skeletal muscle (Thomsen disease, |
| | autosomal dominant) |
| CAV3 | Caveolin 3 |
| HSPG2 | Perlecan |
| ATP2A1 | ATPase, Ca++ transporting, fast twitch 1 |

### Ion Channel Muscle Diseases

| Gene | protein |
|---|---|
| CLCN1 | Chloride channel 1, skeletal muscle (Thomsen disease, |
| | autosomal dominant) |
| SCN4A | Sodium channel, voltage-gated, type IV, alpha |
| SCN5A | Voltage-gated sodium channel type V alpha |
| CACNA1S | Calcium channel, voltage-dependent, L type, alpha 1S |
| | subunit |
| CACNA1A | Calcium channel, voltage-dependent. P/Q type, alpha 1A |
| | subunit |
| KCNE3 | Potassium voltage-gated channel, Isk-related family, |
| | member 3 |
| KCNA1 | Potassium voltage-gated channel, shaker-related subfamily, |
| | member 1 |
| KCNJ18 | Kir2.6 (inwardly rectifying potassium channel 2.6) |
| KCNJ2 | Potassium inwardly-rectifying channel J2 |
| KCNH2 | Voltage-gated potassium channel, subfamily H, member 2 |
| KCNQ1 | Potassium voltage-gated channel. KQT-like subfamily, |
| | member 1 |
| KCNE2 | Potassium voltage-gated channel, Isk-related family, |
| | member 2 |
| KCNE1 | Potassium voltage-gated channel, Isk-related family, |
| | member 1 |

### Malignant Hyperthermia

| Gene | protein |
|---|---|
| RYR1 | Ryanodine receptor 1 (skeletal) |
| CACNA1S | Calcium channel, voltage-dependent, |
| | L type, alpha 1S subunit |

### Metabolic Myopathies

| Gene | protein |
|---|---|
| GAA | Acid alpha-glucosidase preproprotein |
| AGL | Amylo-1,6-glucosidase, 4-alpha-glucanotransferase |
| GBE1 | Glucan (1,4-alpha-), branching enzyme 1 (glycogen |
| | branching enzyme, Andersen disease, glycogen storage |
| | disease type IV) |
| PYGM | Glycogen phosphorylase |
| PFKM | Phosphofructokinase, muscle |
| PHKA1 | Phosphorylase b kinase, alpha submit |
| PGM1 | Phosphoglucomutase 1 |
| GYG1 | Glycogenin 1 |
| GYS1 | Glycogen synthase 3 glycogen synthase 1 (muscle) |
| | glycogen synthase 1 (muscle) |
| PRKAG2 | Protein kinase, AMP-activated, gamma 2 non-catalytic |
| | subunit |
| RBCK1 | RanBP-type and C3HC4-type zinc finger containing 1 |
| | (heme-oxidized IRP2 ubiquitin ligase 1) |
| PGK1 | Phosphoglycerate kinase 1 |
| PGAM2 | Phosphoglycerate mutase 2 (muscle) |
| LDHA | Lactate dehydrogenase A |
| ENO3 | Enolase 3, beta muscle specific |
| CPT2 | Carnitine palmitoyltransferase II |
| SLC22A5 | Solute carrier family 22 member 5 |
| SLC25A20 | Carnitine-acylcarnitine translocase |
| ETFA | Electron-transfer-flavoprotein, alpha polypeptide |
| ETFB | Electron-transfer-flavoprotein, beta polypeptide |
| ETFDH | Electron-transferring-flavoprotein dehydrogenase |
| ACADVL | Acyl-Coenzyme A dehydrogenase, very long chain |
| ABHD5 | Abhydrolase domain containing 5 |
| PNPLA2 | Adipose triglyceride lipase (desnutrin) |
| LPIN1 | Lipin 1 (phosphatidic acid phosphatase 1) |
| PNPLA8 | Patatin-like phospholipase domain containing 8 |

### Hereditary Cardiomyopathies

| Gene | protein |
|---|---|
| MYH6 | Myosin heavy chain 6 |
| MYH7 | Myosin, heavy polypeptide 7, cardiac muscle, beta |
| TNNT2 | Troponin T2, cardiac |
| TPM1 | Tropomyosin 1 (alpha) |
| MYBPC3 | Cardiac myosin binding protein-C |
| PRKAG2 | Protein kinase, AMP-activated, gamma 2 non-catalytic |
| | subunit |
| TNNI3 | Troponin I, cardiac |
| MYL3 | Myosin light chain 3 |
| TTN | Titin |
| MYL2 | Myosin light chain 2 |
| ACTC1 | Actin, alpha, cardiac muscle precursor |
| CSRP3 | Cysteine and glycine-rich protein 3 (cardiac LIM protein) |
| TNNC1 | Slow troponin C |
| VCL | Vinculin |
| MYLK2 | Myosin light chain kinase 2 |
| CAV3 | Caveolin 3 |
| MYOZ2 | Myozenin 2, or calsarcin 1, a Z disk protein |
| JPH2 | Junctophilin-2 |
| PLN | Phospholamban |
| NEXN | Nexilin(F-actin binding protein) |
| ANKRD1 | Ankyrin repeat domain 1 (cardiac muscle) |
| ACTN2 | Actinin alpha2 |
| NDUFAF1 | NADH-ubiquinone oxidoreductase 1 alpha subcomplex |
| TSFM | Ts translation elongation factor, mitochondrial |
| AARS2 | Alanyl-tRNA synthetase 2, mitochondrial |
| MRPL3 | Mitochondrial ribosomal protein L3 |
| COX15 | COX15 homolog, cytochrome c oxidase assembly protein |
| | (yeast) |
| MTO1 | Mitochondrial tRNA translation optimization 1 |
| MRPL44 | Mitochondrial ribosomal protein L44 |
| LMNA | Lamin A/C |
| LDB3 | LIM domain binding 3 |
| SCN5A | Voltage-gated sodium channel type V alpha |
| DES | Desmin |
| EYA4 | Eyes absent 4 |
| SGCD | Delta-sarcoglycan |
| TCAP | Telethonin |
| ABCC9 | ATP-binding cassette, sub-family C (member 9) |
| TMPO | Lamina-associated polypeptide 2 |
| PSEN2 | Presenilin 2 |
| CRYAB | Crystallin, alpha B |
| FKTN | Fukutin |
| TAZ | Tafazzin |
| DMD | Dystrophin |
| LAMA4 | Laminin alpha 4 |
| ILK | Integrin-linked kinase |
| MYPN | Myopalladin |
| RBM20 | RNA binding motif protein 20 |
| SYNE1 | Spectrin repeat containing, nuclear envelope 1 (nesprin 1) |
| MURC | Muscle-related coiled-coil protein |
| DOLK | Dolichol kinase |
| GATAD1 | GATA zinc finger domain containing 1 |
| SDHA | succinate dehydrogenase complex, subunit A, |
| | flavoprotein (Fp) |
| GAA | Acid alpha-glucosidase preproprotein |
| DTNA | Dystrobrevin, alpha |
| FLNA | Filamin A, alpha (actin binding protein 280) |
| TGFB3 | Transforming growth factor, beta 3 |
| RYR2 | Ryanodine receptor 2 |
| TMEM43 | Transmembrane protein 43 |
| DSP | Desmoplakin |
| PKP2 | Plakophilin 2 |
| DSG2 | Desmoglein 2 |
| DSC2 | Desmocollin 2 |
| JUP | Junction plakoglobin |
| CASQ2 | Calsequestrin 2 (cardiac muscle) |
| KCNQ1 | Potassium voltage-gated channel, KQT-like subfamily, |
| | member 1 |
| KCNH2 | Voltage-gated potassium channel, subfamily H, member 2 |
| ANK2 | Ankyrin 2 |
| KCNE1 | Potassium voltage-gated channel, Isk-related family, |
| | member 1 |
| KCNE2 | Potassium voltage-gated channel. Isk-related family, |
| | member 2 |
| KCNJ2 | Potassium inwardly-rectifying channel J2 |
| CACNA1C | Calcium channel, voltage-dependent, L type, alpha 1C |
| | subunit |
| SCN4B | Sodium channel, voltage-gated, type IV, beta subunit |
| AKAP9 | A kinase (PRKA) anchor protein (yotiao) 9 |
| SNTA1 | Syntrophin, alpha 1 |
| KCNJ5 | Potassium inwardly-rectifying channel, subfamily J, |
| | member 5 |
| NPPA | Natriuretic peptide precursor A |
| KCNA5 | Potassium voltage-gated channel, shaker-related subfamily, |
| | member 5 |
| GJA5 | Connexin 40 |
| SCN1B | Sodium channel, voltage-gated, type I, beta subunit |
| SCN2B | Sodium channel, voltage-gated, type II, beta subunit |
| NUP155 | Nucleoporin 155 kDa |
| GPD1L | Glycerol-3-phosphate dehydrogenase 1-like |
| CACNB2 | Calcium channel, voltage-dependent, beta 2 subunit |
| KCNE3 | Potassium voltage-gated channel, Isk-related family, |
| | member 3 |
| SCN3B | Sodium channel, voltage-gated, type III, beta subunit |
| HCN4 | Hyperpolarization activated cyclic nucleotide-gated |
| | potassium channel 4 |

### Congenital Myasthenic Syndromes

| Gene | protein |
|---|---|
| CHRNA1 | Cholinergic receptor, nicotinic, alpha polypeptide 1 |
| CHRNB1 | Cholinergic receptor, nicotinic, beta 1 muscle |
| CHRND | Cholinergic receptor, nicotinic, delta |
| CHRNE | Cholinergic receptor, nicotinic, epsilon |
| RAPSN | Rapsyn |
| CHAT | Choline acetyltransferase isoform |
| COLQ | Acetylcholinesterase collagen-like tail subunit |
| MUSK | muscle, skeletal, receptor tyrosine kinase |
| DOK7 | Docking protein 7 |
| AGRN | Agrin |
| GFPT1 | Glutamine-fructose-6-phosphate transaminase 1 |
| DPAGT1 | Dolichyl-phosphate (UDP-N-acetylglucosamine) N- |
| | acetylglucosaminephosphotransferase 1 (GlcNAc-1-P |
| | transferase) |
| LAMB2 | Laminin, beta 2 (laminin S) |
| SCN4A | Sodium channel, voltage-gated, type IV, alpha |
| CHRNG | Cholinergic receptor, nicotinic, gamma polypeptide |
| PLEC | plectin |
| ALG2 | Alpha-1,3/1,6-mannosyltransferase |
| ALG14 | UDP-N-acetylglucosaminyltransferase |
| SYT2 | Synaptotagmin II |
| PREPL | Prolyl endopeptidase-like |

### Motor Neuron Diseases

| Gene | protein |
|---|---|
| SMN1 | Survival of motor neuron 1, telomeric |
| IGHMBP2 | Immunoglobulin mu binding protein 2 |
| PLEKHG5 | Pleckstrin homology domain containing, family G (with |
| | RhoGef domain) member 5 |
| HSPB8 | Heat shock 27 kDa protein 8 |
| HSPB1 | Heat shock 27 kDa protein 1 |
| HSPB3 | Heat shock 27 kDa protein 3 |
| AARS | Alanyl-tRNA synthetase |
| GARS | Glycyl-tRNA synthetase |
| BSCL2 | Seipin |
| REEP1 | Receptor accessory protein 1 |
| SLC5A7 | Solute carrier family 5 (sodium/choline cotransporter), |
| | member 7 |
| DCTN1 | Dynactin 1 |
| UBA1 | Ubiquitin-activating enzyme 1 |
| ATP7A | ATPase, Cu++ transporting, alpha polypeptide |
| DNAJB2 | DnaJ (Hsp40) homolog, subfamily B, member 2 |
| TRPV4 | Transient receptor potential cation channel, subfamily V, |
| | member 4 |
| DYNC1H1 | Dynein, cytoplasmic 1, heavy chain 1 |
| BICD2 | Bicaudal D homolog 2 (Drosophila) |
| FBXO38 | F-box protein 38 |
| ASAH1 | N-acylsphingosine amidohydrolase (acid ceramidase) 1 |
| VAPB | Vesicle-associated membrane protein-associated protein B |
| | and C |
| EXOSC8 | Exosome component 8 |
| SOD1 | Superoxide dismutase 1, soluble |
| ALS2 | Alsin |
| SETX | Senataxin |
| FUS | Fusion (involved in t(12; 16) in malignant liposarcoma) |
| ANG | Angiogenin |
| TARDBP | TAR DNA binding protein |
| FIG4 | Sac domain-containing inositol phosphatase 3 |
| OPTN | Optineurin |
| ATXN2 | Ataxin 2 |
| VCP | Valosin-containing protein |
| UBQLN2 | Ubiquilin 2 |
| SIGMAR1 | Sigma non-opioid intracellular receptor 1 |
| CHMP2B | Charged multivesicular body protein 2B |
| PFN1 | Profilin 1 |
| MATR3 | Matrin 3 |
| NEFH | Neurofilament, heavy polypeptide |
| PRPH | Peripherin |
| C9orf72 | Chromosome 9 open reading frame 72 |
| CHCHD10 | Coiled-coil-helix-coiled-coil-helix domain containing 10 |
| SQSTM1 | Sequestosome 1 |
| AR | Androgen receptor |
| GLE1 | GLE1 RNA export mediator homolog (yeast) |
| ERBB3 | V-erb-b2 erythroblastic leukemia viral oncogene homolog 3 |
| | (avian) |
| PIP5K1C | Phosphatidylinositol-4-phosphate 5-kinase, type I, gamma |
| EXOSC3 | Exosome component 3 |
| VRK1 | Vaccinia related kinase 1 |
| SLC52A3 | Solute carrier family 52, riboflavin transporter, member 3 |
| SLC52A2 | Solute carrier family 52, riboflavin transporter, member 2 |
| HEXB | Hexosaminidase B |

### Hereditary Motor and Sensory Neuropathies

| Gene | Protein |
|---|---|
| PMP22 | Peripheral myelin protein 22 |
| MPZ | Myelin protein zero |
| LITAF | Lipopolysaccharide-induced TNF factor |
| EGR2 | Early growth response 2 protein |
| NEFL | Neurofilament, light polypeptide 68 kDa |
| HOXD10 | Homeobox D10 |
| ARHGEF10 | Rho guanine nucleotide exchange factor 10 |
| FBLN5 | Fibulin 5 (extra-cellular matrix) |
| DNM2 | Dynamin 2 |
| YARS | Tyrosyl-tRNA synthetase |
| INF2 | Inverted formin 2 |
| GNB4 | Guanine nucleotidebinding protein, beta-4 |
| GDAP1 | Ganglioside-induced differentiation-associated protein 1 |
| MTMR2 | Myotubularin-related protein 2 |
| SBF2 | SET binding factor 2 |
| SBF1 | SET binding factor 1 |
| SH3TC2 | KIAA1985 protein |
| NDRG1 | N-myc downstream regulated gene 1 |
| PRX | Periaxin |
| HK1 | Hexokinase 1 |
| FGD4 | Actin-filament binding protein Frabin |
| FIG4 | Sac domain-containing inositol phosphatase 3 |
| SURF1 | surfeit 1 |
| GJB1 | Gap junction protein, beta 1, 32 kDa (connexin 32) |
| AIFM1 | Apoptosis-inducing factor, mitochondrionassociated 1 |
| PRPS1 | Phosphoribosyl pyrophosphate synthetase 1 |
| PDK3 | Pyruvate dehydrogenase kinase, isoenzyme 3 |
| KIF1B | Kinesin family member 1B |
| MFN2 | Mitofusin 2 |
| RAB7A | RAB7, member RAS oncogene family |
| TRPV4 | Transient receptor potential cation channel, subfamily V, |
| | member 4 |
| GARS | Glycyl-tRNA synthetase |
| HSPB1 | Heat shock 27 kDa protein 1 |
| HSPB8 | Heat shock 27 kDa protein 8 |
| AARS | Alanyl-tRNA synthetase |
| DYNC1H1 | Dynein, cytoplasmic 1, heavy chain 1 |
| LRSAM1 | leucine rich repeat and sterile alpha motif containing 1 |
| DHTKD1 | dehydrogenase E1 and transketolase domain containing 1 |
| TRIM2 | Tripartite motif containing 2 |
| TFG | TRK-fused gene |
| MARS | methionyl-tRNA synthetase |
| KIF5A | Kinesin family member 5A |
| LMNA | Lamin A/C |
| MED25 | Mediator complex subunit 25 |
| DNAJB2 | DnaJ (Hsp40) homolog, subfamily B, member 2 |
| HINT1 | Histidine triad nucleotide binding protein 1 |
| KARS | Lysyl-tRNA synthetase |
| PLEKHG5 | Pleckstrin homology domain containing, family G (with |
| | RhoGef domain) member 5 |
| COX6A1 | Cytochrome c oxidase subunit VIa polypeptide 1 |
| IGHMBP2 | Immunoglobulin mu binding protein 2 |
| SPTLC1 | Serine palmitoyltransferase subunit 1 |
| SPTLC2 | Serine palmitoyltransferase long chain base subunit 2 |
| ATL1 | Atlastin GTPase 1 |
| KIF1A | Kinesin family member 1A |
| WNK1 | WNK lysine deficient protein kinase 1 |
| IKBKAP | Inhibitor of kappa light polypeptide gene enhancer in |
| | B-cells, kinase complex-associated protein |
| NGF | Nerve growth factor (beta polypeptide) |
| DNMT1 | DNA (cytosine-5)-methyltransferase 1 |
| SLC12A6 | Potassium chloride cotransporter KCC3 |
| GJB3 | Gap junction protein, beta 3, 31 kDa (=connexin 31) |
| sept-09 | Septin 9 |
| GAN | Gigaxonin |
| CTDP1 | CTD phosphatase subunit 1 |
| VRK1 | Vaccinia related kinase 1 |

### Hereditary Paraplegia

| Gene | |
|---|---|
| symbol | protein |
| ATL1 | Atlastin |
| SPAST | Spastin |
| NIPA1 | Non-imprinted in Prader-Willi/Angelman syndrome 1 |
| KIAA0196 | Strumpellin |
| KIF5A | Kinesin family member 5A |
| RTN2 | Reticulon 2 |
| HSPD1 | Heat shock 60 kDa protein 1 (chaperonin) |
| BSCL2 | Seipin |
| REEP1 | Receptor accessory protein 1 |
| ZFYVE27 | Protrudin |
| SLC33A1 | Solute carrier family 33 (acetyl- CoA transporter) |
| CYP7B1 | Cytochrome P450, family 7, subfamily B, polypeptide 1 |
| SPG7 | Paraplegin |
| SPG11 | Spatacsin |
| ZFYVE26 | Spastizin |
| ERLIN2 | ER lipid raft associated 2 |
| SPG20 | Spartin |
| SPG21 | Maspardin |
| B4GALNT1 | beta-1,4-N-acetyl-galactosaminyl transferase 1 |
| DDHD1 | DDHD domain containing 1 |
| KIF1A | Kinesin family member 1A |
| FA2H | Fatty acid 2-hydroxylase |
| PNPLA6 | Patatin-like phospholipase domain containing 6 |
| C19orf12 | chromosome 19 open reading frame 12 |
| GJC2 | gap junction protein, gamma 2, 47 kDa |
| NT5C2 | 5′-nucleotidase, cytosolic II |
| GBA2 | glucosidase, beta (bile acid) 2 |
| AP4B1 | adaptor-related protein complex 4, beta 1 subunit |
| AP5Z1 | Hypothetical protein LOC9907 |
| TECPR2 | tectonin beta-propeller repeat containing 2 |
| AP4M1 | Adaptor-related protein complex 4, mu 1 subunit |
| AP4E1 | Adaptor-related protein complex 5, zeta 1 subunit |
| AP4S1 | adaptor-related protein complex 4, sigma 1 subunit |
| DDHD2 | DDHD domain containing 2 |
| C12orf65 | adaptor-related protein complex 4, sigma 1 subunit |
| CYP2U1 | cytochrome P450, family 2, subfamily U, polypeptide 1 |
| ARL6IP1 | ADP-ribosylation factor-like 6 interacting protein 1 |
| AMPD2 | adenosine monophosphate deaminase 2 |
| ENTPD1 | ectonucleoside triphosphate diphosphohydrolase 1 |
| ALDH3A2 | Aldehyde dehydrogenase 3A2 |
| ALS2 | Alsin |
| L1CAM | L1 cell adhesion molecule |
| PLP1 | Proteolipid protein 1 |
| MTPAP | mitochondrial poly(A) polymerase |
| AFG3L2 | AFG3 ATPase family gene 3-like 2 (S. cerevisiae) 1 |
| SACS | Sacsin |

### Other Neuromuscular Disorders

| Gene | protein |
|---|---|
| TOR1A | Torsin A |
| SGCE | Sarcoglycan, epsilon |
| IKBKAP | Inhibitor of kappa light polypeptide gene enhancer in B- |
| | cells, kinase complex-associated protein |
| TTR | Transthyretin (prealbumin, amyloidosis type I) |
| KIF21A | Kinesin family member 21A |
| PHOX2A | Paired-like aristaless homeobox protein 2A |
| TUBB3 | Tubulin, beta 3 |
| TPM2 | Tropomyosin 2 (beta) |
| MYH3 | Myosine, heavy chain 3, skeletal muscle, embryonic |
| TNNI2 | Troponin I, type 2 |
| TNNT3 | Troponin T3, skeletal |
| SYNE1 | Spectrin repeat containing, nuclear envelope 1 (nesprin 1) |
| MYH8 | Myosin heavy chain, 8, skeletal muscle, perinatal |
| POLG | Polymerase (DNA directed), gamma |
| SLC25A4 | Mitochondrial carrier; adenine nucleotide translocator |
| C10orf2 | chromosome 10 open reading frame 2 |
| POLG2 | Mitochondrial DNA polymerase, accessory subunit |
| RRM2B | Ribonucleotide reductase M2 B (TP53 inducible) |
| TK2 | Thymidine kinase 2, mitochondrial |
| SUCLA2 | Succinate-CoA ligase, ADP-forming, beta subunit |
| OPA1 | optic atrophy 1 |
| STIM1 | Stromal interaction molecule 1 |
| ORAI1 | ORAI calcium release-activated calcium modulator 1 |
| PUS1 | Pseudouridylate synthase 1 |
| CHCHD10 | Coiled-coil-helix-coiled-coil-helix domain containing 10 |
| CASQ1 | Calsequestrin 1 (fast-twitch, skeletal muscle) |
| YARS2 | tyrosyl-tRNA synthetase 2, mitochondrial |

Any one of the above listed genes may be targeted in replacement gene therapy, wherein the gene of interest is a functional version of the deficient or mutated gene.

Alternatively, the above listed genes may be used as target for gene editing. Gene editing is used to correct the sequence of a mutated gene or modify the expression or regulation of a deficient/abnormal gene so that a functional gene is expressed in muscle cells. In such cases, the gene of interest is chosen from those encoding therapeutic RNAs such as interfering RNAs, guide RNAs for genome editing and antisense RNAs capable of exon skipping, wherein the therapeutic RNAs target the preceding list of genes. Tools such as CRISPR/Cas9 may be used for that purpose.

In some embodiments, the target gene for gene therapy (additive gene therapy or gene editing) is a gene responsible for one of the muscular dystrophies listed above, in particular DMD (DMD, BMD genes); LGMDs (CAPN3 gene and others); Facio-scapulo-humeral dystrophies, type 1 (FSHD1A; DUX4 or FRG1 gene) and type 2 (FSHD1B; SMCHD1 gene) and titinopathies (TTN gene).

In some embodiments, the pharmaceutical composition of the invention is for use for treating muscular diseases (i.e., myopathies) or muscular injuries, in particular neuromuscular genetic disorders, with no liver damage, such as for example: Muscular dystrophies, Congenital muscular dystrophies, Congenital myopathies, Distal myopathies, Other myopathies, Myotonic syndromes, Ion Channel muscle diseases, Malignant hyperthermia, Metabolic myopathies, Hereditary Cardiomyopathies, Congenital myasthenic syndromes, Motor Neuron diseases, Hereditary paraplegia, Hereditary motor and sensory neuropathies and other neuromuscular disorders.

Muscular dystrophies include in particular:
Dystrophinopathies, a spectrum of X-linked muscle diseases caused by pathogenic variants in DMD gene, which encodes the protein dystrophin. Dystrophinopathies comprises Duchenne muscular dystrophy (DMD), Becker muscular dystrophy (BMD) and DMD-associated dilated cardiomyopathy;
The Limb-girdle muscular dystrophies (LGMDs) which are a group of disorders that are clinically similar to DMD but occur in both sexes as a result of autosomal recessive and autosomal dominant inheritance. Limb-girdle dystrophies are caused by mutation of genes that encode sarcoglycans and other proteins associated with the muscle cell membrane, which interact with dystrophin. The term LGMD1 refers to genetic types showing dominant inheritance (autosomal dominant), whereas LGMD2 refers to types with autosomal recessive inheritance. Pathogenic variants at more than 50 loci have been reported (LGMDA to LGMD1H; LGMD2A to LGMD2Y).

Calpainopathy (LGMD2A) is caused by mutation of the gene CAPN3 with more than 450 pathogenic variants described;
The Emery-Dreifuss Muscular Dystrophy (EDMD) caused by defects in one of the gene including the EMD gene (coding for emerin), the FHL1 gene and the LMNA gene (encoding lamin A and C);
Nesprin-1 and Nesprin-2 related muscular dystrophy caused by defects in the SYNE1 and SYNE2 gene, respectively; LUMA related muscular dystrophy caused by defects in the TMEM43 gene; LAP1B related muscular dystrophy caused by defects in the TOR1AIP1 gene; and
Facio-scapulo-humeral muscular dystrophy, type 1 (FSHD1A), such as associated with defect in the DUX4 gene (contraction of the D4Z4 macrosatellite repeat in the subtelomeric region of chromosome 4q35) or the FRG1 gene; Facio-scapulo-humeral muscular dystrophy, type 2 (FSHD1B) caused by defects in the SMCHD1 gene.

A specific example of gene editing would be the treatment of Limb-girdle muscular dystrophy 2A (LGMD2A) which is caused by mutations in the calpain-3 gene (CAPN3). Other examples would be the treatment of mutations in the DMD or TNT genes.

Thus, by gene editing or gene replacement a correct version of this gene is provided in muscle cells of affected patients, this may contribute to effective therapies against this disease. Other genetic diseases of the muscle as listed above could be treated by gene replacement or gene editing using the same principle.

Replacement or additive gene therapy may be used to treat cancer, in particular rhabdomyosarcomas. Genes of interest in cancer could regulate the cell cycle or the metabolism and migration of the tumor cells, or induce tumor cell death. For instance, inducible caspase-9 could be expressed in muscle cells to trigger cell death, preferably in combination therapy to elicit durable anti-tumor immune responses.

Gene editing may be used to modify gene expression in muscle cells, in the case of auto-immunity or cancer, or to perturb the cycle of viruses in such cells. In such cases, preferably, the gene of interest is chosen from those encoding guide RNA (gRNA), site-specific endonucleases (TALEN, meganucleases, zinc finger nucleases, Cas nuclease), DNA templates and RNAi components, such as shRNA and microRNA. Tools such as CRISPR/Cas9 may be used for this purpose.

In some embodiments, gene therapy is used for treating diseases affecting other tissues, by expression of a therapeutic gene in muscle tissue. This is useful to avoid expression of the therapeutic gene in the liver, in particular in patients having a concurrent hepatic disorder such as hepatitis. The therapeutic gene encodes preferably a therapeutic protein, peptide or antibody which is secreted from the muscle cells into the blood stream where it can be delivered to other target tissues such as for example the liver. Examples of therapeutic genes include with no limitation: Factor VIII, Factor IX and GAA genes.

The pharmaceutical composition of the invention which comprises AAV vector particles with reduced liver tropism may be administered to patients having concurrent liver disease such as for example hepatitis including viral or toxic hepatitis.

In the context of the invention, a therapeutically effective amount refers to a dose sufficient for reversing, alleviating or inhibiting the progress of the disorder or condition to which such term applies, or reversing, alleviating or inhibiting the progress of one or more symptoms of the disorder or condition to which such term applies.

The effective dose is determined and adjusted depending on factors such as the composition used, the route of administration, the physical characteristics of the individual under consideration such as sex, age and weight, concurrent medication, and other factors, that those skilled in the medical arts will recognize.

In the various embodiments of the present invention, the pharmaceutical composition comprises a pharmaceutically acceptable carrier and/or vehicle.

A “pharmaceutically acceptable carrier” refers to a vehicle that does not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

Preferably, the pharmaceutical composition contains vehicles, which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or suspensions. The solution or suspension may comprise additives which are compatible with viral vectors and do not prevent viral vector particle entry into target cells. In all cases, the form must be sterile and must be fluid to the extent that easy syringe ability exists. It must be stable under the conditions of manufacture and storage and must be preserved against the contaminating action of microorganisms, such as bacteria and fungi. An example of an appropriate solution is a buffer, such as phosphate buffered saline (PBS) or Ringer lactate.

The invention provides also a method for treating a disease affecting muscle tissue in particular skeletal muscle tissue and/or cardiac tissue, comprising: administering to a patient a therapeutically effective amount of the pharmaceutical composition as described above.

The invention provides also a method for treating a disease by expression of a therapeutic gene in muscle tissue, comprising: administering to a patient a therapeutically effective amount of the pharmaceutical composition as described above.

As used herein, the term “patient” or “individual” denotes a mammal. Preferably, a patient or individual according to the invention is a human.

In the context of the invention, the term “treating” or “treatment”, as used herein, means reversing, alleviating or inhibiting the progress of the disorder or condition to which such term applies, or reversing, alleviating or inhibiting the progress of one or more symptoms of the disorder or condition to which such term applies.

The pharmaceutical composition of the present invention, is generally administered according to known procedures, at dosages and for periods of time effective to induce a therapeutic effect in the patient.

The administration may be parenteral, oral, local, or loco-regional. The parenteral administration is advantageously by injection or perfusion, such as e subcutaneous (SC), intramuscular (IM), intravascular such as intravenous (IV), intraperitoneal (IP), intradermal (ID) or else. Preferably, the administration produces a systemic effect in the whole body, i.e., all the muscles of the patient, including the diaphragm and the heart. Preferably, the administration is systemic, more preferably parenteral.

The practice of the present invention will employ, unless otherwise indicated, conventional techniques, which are within the skill of the art. Such techniques are explained fully in the literature.

The invention will now be exemplified with the following examples, which are not limitative, with reference to the attached drawings in which:

### FIGURE LEGENDS

FIG. 1: Design of new hybrid AAV serotypes between AAV9 and AAVrh74.

A. Cap genes (VP1) of AAV9 and AAVrh74 highlighting the sequence of the variable region. The variable region N-term and C-term sequences are SEQ ID NO: 37 and SEQ ID NO: 38 for AAV9 and SEQ ID NO: 39 and SEQ ID NO: 40 for AAVrh74. B. Hybrid AAV9-rh74 and hybrid AAVrh74-9 Cap genes (VP1).

FIG. 2: Productions of new hybrid AAV serotypes between AAV9 and AAVrh74

AAV9-rh74 and AAVrh74-9 hybrid serotypes and controls (AAV9, AAVrh74) were produced in HEK293T cells. Viral genomes were quantified by Taqman real-time PCR. Error bars represent SEM.

FIG. 3: Design of biodistribution study.

Vg: viral genome.

FIG. 4: Quantification of transgene expression in muscles and organs following systemic administration of AAV hybrid serotypes.

Luciferase expression was quantified in skeletal muscles (A and B) and organs (C and D) of mice injected (with low dose=2 E10 vg/mouse (A and C) or high dose=1 E11 vg/mouse (B and D) of AAV9-rh74 and AAVrh74-9 hybrid serotypes and controls (AAV9, AAVrh74). Error bars represent SEM. TA: Tibialis anterior. Pso: Psoas. Qua: Quadriceps. Dia: Diaphragm. RLU: relative light units.

### EXAMPLE 1: DESIGN AND PRODUCTION OF HYBRID rAAV SEROTYPE VECTORS WITH AAV9-rh74 AND rh74-AAV9 CAPSIDS

### 1. Material and Methods

### Plasmid Construction for New Serotypes

To construct a plasmid containing AAV2 Rep sequence and Hybrid Cap 9-rh74, a fragment of 1029 nt, containing the highly variable part of AAV-rh74 Cap flanked with AAV9 Cap sequence fragments and restriction sites BsiWI in 5′ and Eco47III in 3′, was synthesized (GENEWIZ). This fragment was then inserted using the mentioned restriction sites in the plasmid pAAV2-9, which contains AAV2 Rep and AAV9 Cap, to replace the AAV9 Cap corresponding sequence.

To construct a plasmid containing AAV2 Rep sequence and Hybrid Cap rh74-9, a fragment of 2611 nt, containing the highly variable part of AAV-9 Cap flanked with the rest of AAV_rh74 Cap sequence, a part of AAV2 Rep sequence and restriction sites, HindIII in 5′ and PmeI in 3′, was synthesized (GENEWIZ). This fragment was then inserted using the mentioned restriction sites in the plasmid pAAV2-9, which contains AAV2 Rep and AAV9 Cap, to replace the full AAV9 Cap sequence.

### AAV Production

Two protocols, corresponding to two scales of production, were used in this study. In the miniscale condition, adherent HEK293 are grown in DMEM added with 10% fetal bovine serum (FBS), in multiwell-6 plates. In the upper scale condition, HEK293T are grown in suspension in 250 mL of serum-free medium. The cells are transfected with 3 plasmids: i) a transgene plasmid, containing AAV2 ITRs flanking an expression cassette coding for the firefly luciferase, ii) the helper plasmid pXX6, containing adenoviral sequences necessary for AAV production, and iii) a plasmid containing AAV Rep and Cap genes, defining the serotype of AAV. Two days after transfection, the cells are lysed to liberate the AAV particles. The viral lysate is purified through two rounds of Cesium Chloride density gradient ultracentrifugation followed by dialysis or by affinity chromatography. Viral genomes are quantified by a TaqMan real-time PCR assay using primers and probes corresponding to the ITRs of the AAV vector genome (Rohr et al., J. Virol. Methods, 2002, 106, 81-88).

### 2. Results

### Design of New Serotypes

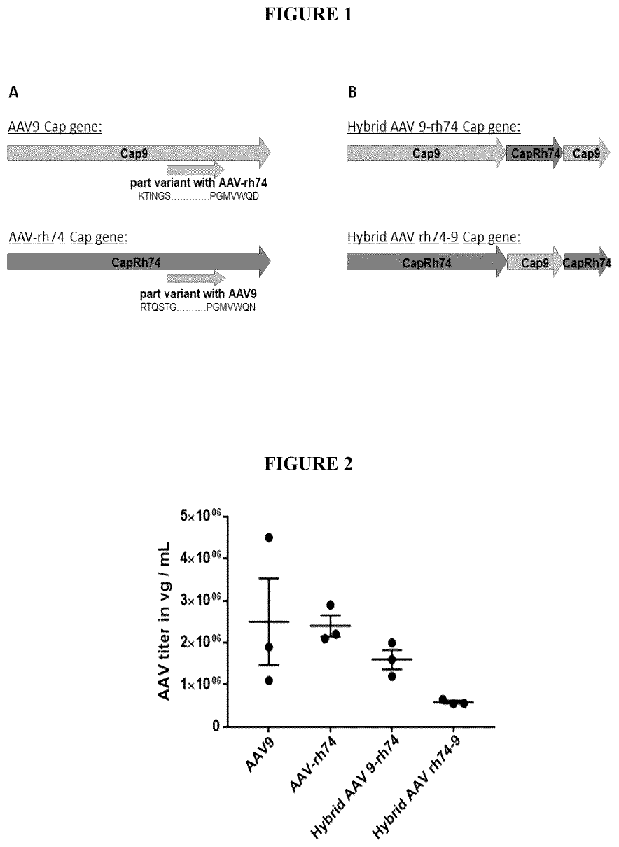

The amino acid sequences of AAV9 (SEQ ID NO: 1) and AAV-rh74 (SEQ ID NO: 2) VP1 protein (encoded by the Cap genes) were aligned using Blastp, and a highly variable region was detected, ranging from amino acid position 449 to position 609 in AAV9 Cap, and from position 450 to position 611 in AAV-rh74 Cap (FIG. 1A). Then two new Cap genes (SEQ ID NO: 5 and SEQ ID NO: 7) were constructed by replacement of the highly variable region of each serotype by the other (FIG. 1B). These two hybrid Cap genes were inserted into a plasmid containing the AAV2 Rep sequence, allowing production of recombinant AAV particles. The new serotypes were named “Hybrid AAV 9-rh74” (SEQ ID NO: 3) for the one containing AAV9 cap sequence for its major part, and the AAV-rh74 highly variable part, and “Hybrid AAV rh74-9” (SEQ ID NO: 4) for the one containing AAVrh74 Cap sequence for its major part, and the AAV9 highly variable part.

### Production of the New Hybrid AAV Serotypes

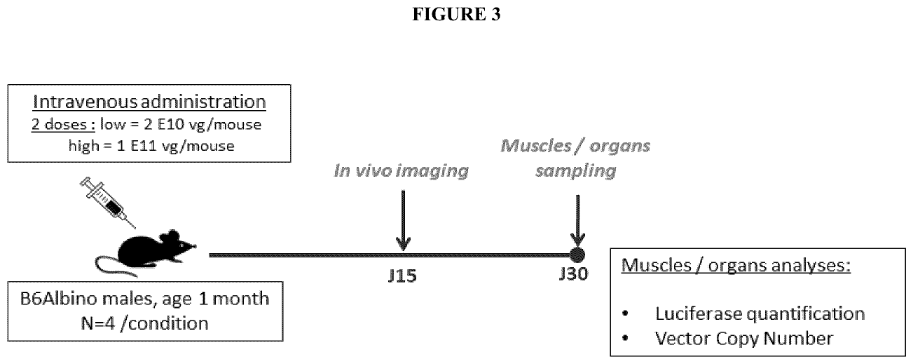

AAV production was performed at two different scales with the new hybrid serotypes and controls (2 mL in 6-well plate or a 250 mL culture in suspension). As shown in FIG. 2, the new hybrid serotypes can be produced with a yield suitable for gene transfer applications.

### EXAMPLE 2: BIODISTRIBUTION STUDY OF HYBRID AAV SEROTYPE VECTORS WITH AAV9-RH74 AND AAVrh74-9 CAPSIDS

### 1. Material and Methods

### In Vivo Experiments

The AAV vectors were administered to one month-old B6Albino male mice, by intravenous injection in the tail vein. Two doses were assessed, a low dose of 2 E10 (2.1010) viral genomes (vg)/mouse, and a high dose of 1 E11 (1011) vg/mouse. Fifteen days after injection, luciferase imaging was performed using IVIS Lumina device (PERKIN ELMER) on mice previously anesthetized (ketamine+xylazine) and injected intraperitonally by luciferin. Thirty days after injection, mice were sacrificed and skeletal muscles and organs were sampled and frozen in liquid nitrogen.

### Molecular Analysis

Samples were homogenized in Lysis buffer [Tris-base 25 mM, MgCl₂ 8 mM, DTT 1 mM, EDTA 1 mM, glycerol 15%, Triton X-100 0.2%] supplemented with Protease Inhibitor Cocktail (Roche). Luciferase expression quantification was performed on sample lysates using Enspire multimode plate reader (Roche), in Assay Buffer [Tris-base 25 mM, MgCl₂ 8 mM, DTT 1 mM, EDTA 1 mM, glycerol 15%, ATP 2 mM] extemporaneously supplemented with luciferin at 83 μM. The total amount of protein in samples was measured using Pierce BCA protein assay kit (Thermo Fisher). The result of luciferase luminescence was normalized by the total protein amount.

For quantification of viral genomes in samples (VCN for Vector Copy Number), DNA was extracted from samples using NucleoSpin Tissue (Macherey-Nagel). Real-time PCR was performed on 100 ng of DNA, using the same protocol as described above for AAV vectors titration. Exon Mex5 of titin gene was amplified in the same experiment to be used as genomic control.

### 2. Results

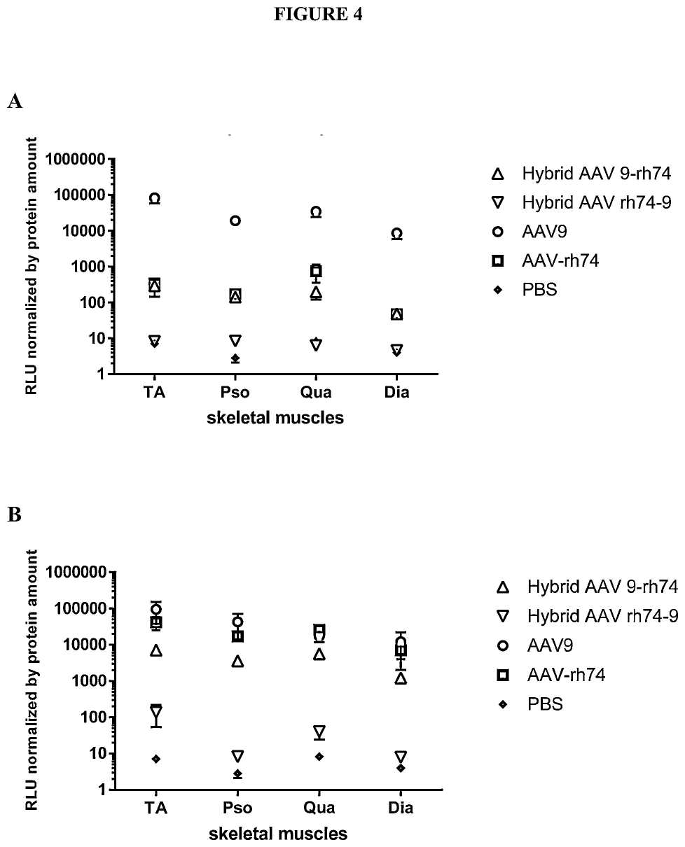

To assess the biodistribution of the new serotypes, Hybrid AAV and control vectors containing an expression cassette encoding the luciferase reporter gene under the control of the ubiquitous CMV promoter were produced. The vectors were administered to mice at two doses (low dose=2 E10 vg/mouse; high dose=1 E11 vg/mouse), by systemic injection. Whole body imaging was performed 15 days after administration, and different skeletal muscles and organs were sampled after one month of expression (FIG. 3).

After sampling, luciferase expression in muscles and other organs was quantified then normalized by total protein amount in sample, in different skeletal muscles and organs.

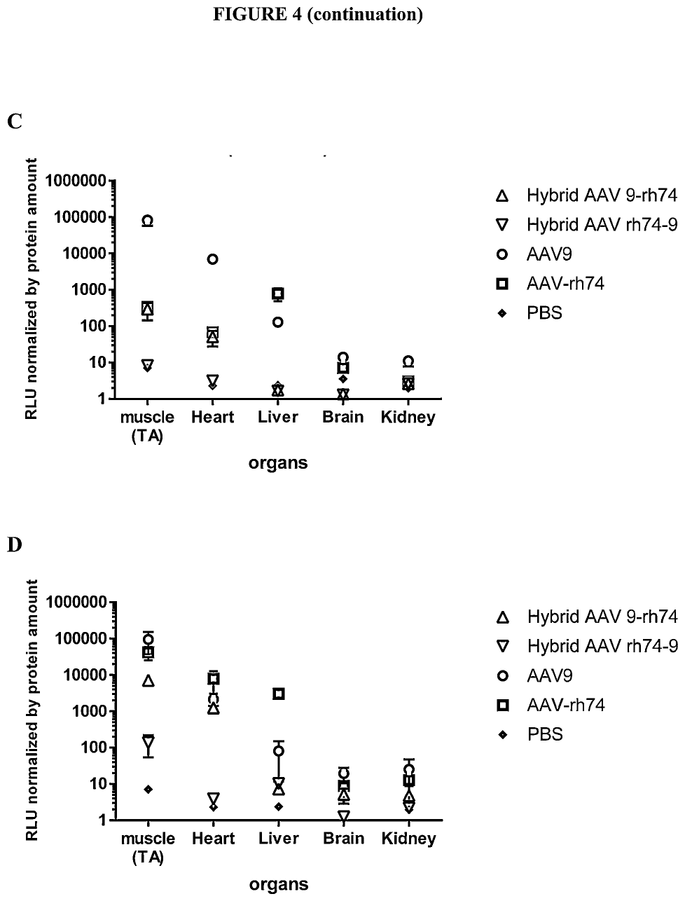

In muscle, hybrid AAV 9-74 allowed a good level of transgene expression in all tested muscles including skeletal and cardiac muscles, similarly to AAVrh74 (FIGS. 4A to 4D). Surprisingly, both hybrids have a drastically reduced transgene expression in liver, compared to the high level of transgene expression of the AAV9 or AAV-rh74 controls (FIGS. 4C and 4D).

Two new serotypes were generated using a combination of AAV9 and AAV-rh74, two serotypes that efficiently infect the muscle tissue but also the liver. The resulting hybrids show gene transfer in skeletal muscle, without efficient transduction of the liver. These hybrid serotypes are therefore of interest when transduction of skeletal muscle but not liver is needed.

### EXAMPLE 3: PEPTIDE-MODIFIED HYBRID AAV9-rh74 SEROTYPE VECTOR

The sequence of the hybrid Cap9-rh74 was modified with a peptide to increase hybrid AAV 9-rh74 serotype vector tropism for muscle tissue. AAV capsid modification was performed according to Kienle EC (Dissertation for the degree of Doctor of natural Sciences, Combined Faculties for the Natural Sciences and for Mathematics of the Ruperto-Carola University of Heidelberg, Germany, 2014) using a peptide as disclosed in Michelfelder et al. (PLoS ONE, 2009, 4, e5122). Briefly, the hexapeptide QQNAAP (SEQ ID NO: 41) present in the VP1 of the hybrid Cap9-rh74 (positions 587 to 592 of SEQ ID NO: 3) is mutated to the octapeptide GQSGAQAA (SEQ ID NO: 42) and peptide P1 (RGDLGLS; SEQ ID NO: 12) is inserted between glycine at position 4 and alanine at position 5. The hybrid Cap9-rh74 modified with peptide P1 has the amino acid sequence SEQ ID NO: 9 and the corresponding coding sequence is SEQ ID NO: 10. Vectors are produced by triple transfection in HEK293 cells grown in suspension and purified by affinity chromatography as described in example 1. Vectors are injected in mice, one month after the injection mice are sacrificed and tissues collected to evaluate the biodistribution as described in example 2. The expected effect of this modification is the increase of the quantity of vector in muscle tissues.

1. A recombinant adeno-associated virus (AAV) capsid protein, which is a hybrid between AAV serotype 9 (AAV9) and AAV serotype 74 (AAVrh74) capsid proteins, wherein said recombinant hybrid AAV capsid protein has a reduced liver tropism compared to the parent AAV9 and AAVrh74 capsid proteins.

2. The recombinant hybrid AAV capsid protein according to claim 1, which has a muscle tropism similar to that of the parent AAV9 and/or AAVrh74 capsid proteins.

3. The recombinant hybrid AAV capsid protein according to claim 1 or claim 2, which results from the replacement of a variable region in the AAV9 or AAVrh74 capsid sequence with the corresponding variable region of the other AAV serotype capsid sequence,
wherein the variable region of AAV9 capsid corresponds to the sequence situated from any one of positions 331 to 493 to any one of positions 556 to 736 in AAV9 capsid of SEQ ID NO: 1 or a fragment of at least 10, 15, 20, 25, 30, 35, 40, 45, 50, 55 or 60 consecutive amino acids of the sequence situated from positions 493 to 556 in AAV9 capsid of SEQ ID NO: 1, and
the variable region of AAVrh74 capsid corresponds to the sequence situated from any one of positions 332 to 495 to any one of positions 558 to 738 in AAVrh74 capsid of SEQ ID NO: 2 or a fragment of at least 10, 15, 20, 25, 30, 35, 40, 45, 50, 55 or 60 consecutive amino acids of the sequence situated from positions 495 to 558 in AAVrh74 capsid of SEQ ID NO: 2.

4. The recombinant hybrid protein according to claim 3, wherein the recombinant hybrid AAV capsid protein results from the replacement of the variable region corresponding to the sequence situated from positions 449 to 609 in AAV9 capsid of SEQ ID NO: 1 or from positions 450 to 611 in AAVrh74 capsid of SEQ ID NO: 2 with the corresponding variable region of the other AAV serotype capsid sequence.

5. The recombinant hybrid AAV capsid protein according to any one of claims 1 to 4, which comprises a sequence selected from the group consisting of the sequences SEQ ID NO: 3 and SEQ ID NO: 4 and the sequences having at least 85%, 90%, 95%, 97%, 98% or 99% identity with said sequences, preferably which comprises a sequence selected from the group consisting of the sequences of SEQ ID NO: 3 and the sequences having at least 85%, 90%, 95%, 97%, 98% or 99% identity with said sequence; more preferably which comprises the sequence of SEQ ID NO: 3.

6. The recombinant hybrid AAV capsid protein according to any one of claims 1 to 5, which comprises the insertion of a peptide which increases the targeting of skeletal or cardiac muscle tissue by AAV vectors.

7. The recombinant hybrid AAV capsid protein according to claim 6, wherein said peptide comprises a sequence selected from the group consisting of SEQ ID NO: 12 to 34.

8. The recombinant hybrid AAV capsid protein according to claim 6 or 7, which comprises a sequence selected from the group consisting of SEQ ID NO: 9 and the sequences having at least 85%, 90%, 95%, 97%, 98% or 99% identity with said sequence.

9. The recombinant hybrid AAV capsid protein according to any one of claims 1 to 8, which is a hybrid VP1, VP2 or VP3 protein.

10. A recombinant chimeric AAV capsid protein, which is selected from the group consisting of:
a chimeric VP1 protein comprising: (i) a VP1-specific N-terminal region having a sequence from natural or artificial AAV serotype other than AAV9 and AAVrh74, (ii) a VP2-specific N-terminal region having a sequence from AAV9, AAVrh74 or natural or artificial AAV serotype other than AAV9 and AAVrh74, and (iii) a VP3 C-terminal region having the sequence of a hybrid VP3 protein according to claim 6, and
a chimeric VP2 protein comprising: (i) a VP2-specific N-terminal region having a sequence from natural or artificial AAV serotype other than AAV9 and AAVrh74, and (ii) a VP3 C-terminal region having the sequence of a hybrid VP3 protein according to claim 6.

11. A polynucleotide encoding the recombinant hybrid AAV capsid protein according to any one of claims 1 to 9 or the recombinant chimeric AAV capsid protein according to claim 10, in expressible form, and eventually further encoding AAV Replicase protein in expressible form.

12. A recombinant plasmid comprising the polynucleotide of claim 11.

13. An AAV vector particle packaging a gene of interest, which comprises the hybrid recombinant AAV capsid protein according to any one of claims 1 to 9, and/or the recombinant chimeric AAV capsid protein according to claim 10, and eventually also at least one AAV capsid protein from natural or artificial AAV serotype other than AAV9 and AAVrh74.

14. The AAV vector particle according to claim 13, wherein the gene of interest is selected from the group consisting of:
(i) therapeutic genes;
(ii) genes encoding therapeutic proteins or peptides such as therapeutic antibodies or antibody fragments and genome editing enzymes; and
(iii) genes encoding therapeutic RNAs such as interfering RNAs, guide RNAs for genome editing and antisense RNAs capable of exon skipping.

15. A pharmaceutical composition comprising a therapeutically effective amount of AAV vector particles according to claim 13 or claim 14.

16. The pharmaceutical composition of claim 15, which is for use as a medicament in gene therapy, preferably for treating genetic diseases, cancer or auto-immune diseases affecting muscle tissues.

17. The pharmaceutical composition for the use according to claim 16, which targets a gene responsible for a neuromuscular genetic disorders selected from the group comprising: Dystrophinopathies, Limb-girdle muscular dystrophies, Facio-scapulo-humeral dystrophies and titinopathies.

18. The pharmaceutical composition for the use according to claim 17, wherein the target gene is selected from the group comprising: DMD, BMD, CAPN3, DUX4, FRG1, SMCHD1 and TTN genes.


SEQUENCE LISTING (42 sequences)

SEQ ID NO: 1 - protein, 736 aa, adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 2 - protein, 738 aa, adeno-associated virus rh74
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 3 - protein, 737 aa, synthetic construct
  REGION 1..737: note = synthetic polypeptide AAV9-rh74 hybrid capsid
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 4 - protein, 737 aa, synthetic construct
  REGION 1..737: note = synthetic polypeptide AAVrh74-9 hybrid capsid
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 5 - DNA, 2214 bp, synthetic construct
  misc_feature 1..2214: note = synthetic polynucleotide encoding AAV9-rh74 hybrid capsid
  source 1..2214: mol_type = other DNA
  CDS 1..2211
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcacggactcaaagcacgggcggtactgcaggaactcagcagttgctattttctcaggccgggcctaacaacatgtcggctcaggccaagaactggctacccggtccctgctaccggcagcaacgcgtctccacgacactgtcgcagaacaacaacagcaactttgcctggacgggtgccaccaagtatcatctgaatggcagagactctctggtgaatcctggcgttgccatggctacccacaaggacgacgaagagcgattttttccatccagcggagtcttaatgtttgggaaacagggagctggaaaagacaacgtggactatagcagcgtgatgctaaccagcgaggaagaaataaagaccaccaacccagtggccacagaacagtacggcgtggtggccgataacctgcaacagcaaaacgccgctcctattgtaggggccgtcaatagtcaaggagccttacctggcatggtgtggcagaacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 6 - protein, 737 aa, synthetic construct; identical to SEQ ID NO: 3
  REGION 1..737: note = Synthetic Construct

SEQ ID NO: 7 - DNA, 2214 bp, synthetic construct
  misc_feature 1..2214: note = synthetic polynucleotide encoding AAVrh74-9 hybrid capsid
  source 1..2214: mol_type = other DNA
  CDS 1..2211
atggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctccaagcgggtgacaatccgtacctgcggtataatcacgccgacgccgagtttcaggagcgtctgcaagaagatacgtcttttgggggcaacctcgggcgcgcagtcttccaggccaaaaagcgggttctcgaacctctgggcctggttgaatcgccggttaagacggctcctggaaagaagagaccggtagagccatcaccccagcgctctccagactcctctacgggcatcggcaagaaaggccagcagcccgcaaaaaagagactcaattttgggcagactggcgactcagagtcagtccccgaccctcaaccaatcggagaaccaccagcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgcacctgggccctgcccacctacaacaaccacctctacaagcaaatctccaacgggacctcgggaggaagcaccaacgacaacacctacttcggctacagcaccccctgggggtattttgacttcaacagattccactgccacttttcaccacgtgactggcagcgactcatcaacaacaactggggattccggcccaagaggctcaacttcaagctcttcaacatccaagtcaaggaggtcacgcagaatgaaggcaccaagaccatcgccaataaccttaccagcacgattcaggtctttacggactcggaataccagctcccgtacgtgctcggctcggcgcaccagggctgcctgcctccgttcccggcggacgtcttcatgattcctcagtacgggtacctgactctgaacaatggcagtcaggctgtgggccggtcgtccttctactgcctggagtactttccttctcaaatgctgagaacgggcaacaactttgaattcagctacaacttcgaggacgtgcccttccacagcagctacgcgcacagccagagcctggaccggctgatgaaccctctcatcgaccagtacttgtactacctgtccaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggaccgggacgtgtacctgcagggtcccatctgggccaagattcctcatacggacggcaactttcatccctcgccgctgatgggaggctttggactgaagcatccgcctcctcagatcctgattaaaaacacacctgttcccgcggatcctccgaccaccttcaatcaggccaagctggcttctttcatcacgcagtacagtaccggccaggtcagcgtggagatcgagtgggagctgcagaaggagaacagcaaacgctggaacccagagattcagtacacttccaactactacaaatctacaaatgtggactttgctgtcaatactgagggtacttattccgagcctcgccccattggcacccgttacctcacccgtaatctgtaa

SEQ ID NO: 8 - protein, 737 aa, synthetic construct; identical to SEQ ID NO: 4
  REGION 1..737: note = Synthetic Construct

SEQ ID NO: 9 - protein, 746 aa, synthetic construct
  REGION 1..746: note = peptide-modified AAV9-rh74 hybrid capsid
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQGQSGRGDLGLSAQAAIVGAVNSQGALPGMVWQNRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 10 - DNA, 2241 bp, synthetic construct
  misc_feature 1..2241: note = synthetic polynucleotide encoding peptide-modified AAV9-rh74 hybrid capsid
  source 1..2241: mol_type = other DNA
  CDS 1..2241
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcacggactcaaagcacgggcggtactgcaggaactcagcagttgctattttctcaggccgggcctaacaacatgtcggctcaggccaagaactggctacccggtccctgctaccggcagcaacgcgtctccacgacactgtcgcagaacaacaacagcaactttgcctggacgggtgccaccaagtatcatctgaatggcagagactctctggtgaatcctggcgttgccatggctacccacaaggacgacgaagagcgattttttccatccagcggagtcttaatgtttgggaaacagggagctggaaaagacaacgtggactatagcagcgtgatgctaaccagcgaggaagaaataaagaccaccaacccagtggccacagaacagtacggcgtggtggccgataacctgcaaggccagagtggccgcggcgatctgggcctgagcgcccaggcggccattgtaggggccgtcaatagtcaaggagccttacctggcatggtgtggcagaacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 11 - protein, 746 aa, synthetic construct; identical to SEQ ID NO: 9
  REGION 1..746: note = Synthetic Construct

SEQ ID NO: 12 - protein, 7 aa, synthetic construct
  REGION 1..7: note = synthetic peptide
RGDLGLS

SEQ ID NO: 13 - protein, 7 aa, synthetic construct
  REGION 1..7: note = synthetic peptide
GGLSGVS

SEQ ID NO: 14 - protein, 7 aa, synthetic construct
  REGION 1..7: note = synthetic peptide
GGLSGDS

SEQ ID NO: 15 - protein, 7 aa, synthetic construct
  REGION 1..7: note = syntehtic peptide
GSVSGSA

SEQ ID NO: 16 - protein, 7 aa, synthetic construct
  REGION 1..7: note = synthetic peptide
EYRDSSG

SEQ ID NO: 17 - protein, 7 aa, synthetic construct
  REGION 1..7: note = synthetic peptide
QMSGGVA

SEQ ID NO: 18 - protein, 7 aa, synthetic construct
  REGION 1..7: note = synthetic peptide
ESGLSQS

SEQ ID NO: 19 - protein, 7 aa, synthetic construct
  REGION 1..7: note = synthetic peptide
ESGIWVA

SEQ ID NO: 20 - protein, 7 aa, synthetic construct
  REGION 1..7: note = synthetic peptide
EEPALRA

SEQ ID NO: 21 - protein, 7 aa, synthetic construct
  REGION 1..7: note = synthetic peptide
APTLGSP

SEQ ID NO: 22 - protein, 7 aa, synthetic construct
  REGION 1..7: note = synthetic peptide
DLGSARA

SEQ ID NO: 23 - protein, 7 aa, synthetic construct
  REGION 1..7: note = synthetic peptide
DGLGRLV

SEQ ID NO: 24 - protein, 7 aa, synthetic construct
  REGION 1..7: note = synthetic peptide
DLRGLAS

SEQ ID NO: 25 - protein, 7 aa, synthetic construct
  REGION 1..7: note = synthetic peptide
DRSPLSL

SEQ ID NO: 26 - protein, 7 aa, synthetic construct
  REGION 1..7: note = synthetic peptide
AISGVGS

SEQ ID NO: 27 - protein, 7 aa, synthetic construct
  REGION 1..7: note = synthetic peptide
DRSGVGS

SEQ ID NO: 28 - protein, 7 aa, synthetic construct
  REGION 1..7: note = synthetic peptide
SISGVGS

SEQ ID NO: 29 - protein, 7 aa, synthetic construct
  REGION 1..7: note = synthetic peptide
SEGRSGV

SEQ ID NO: 30 - protein, 7 aa, synthetic construct
  REGION 1..7: note = synthetic peptide
GEARSRA

SEQ ID NO: 31 - protein, 7 aa, synthetic construct
  REGION 1..7: note = synthetic peptide
GEARISA

SEQ ID NO: 32 - protein, 7 aa, synthetic construct
  REGION 1..7: note = synthetic peptide
SGNSGAA

SEQ ID NO: 33 - protein, 7 aa, synthetic construct
  REGION 1..7: note = synthetic peptide
SSGSGGA

SEQ ID NO: 34 - protein, 7 aa, synthetic construct
  REGION 1..7: note = synthetic peptide
ESGIWVA

SEQ ID NO: 35 - protein, 4 aa, synthetic construct
  REGION 1..4: note = synthetic peptide
GQSG

SEQ ID NO: 36 - protein, 4 aa, synthetic construct
  REGION 1..4: note = synthetic peptide
AQAA

SEQ ID NO: 37 - protein, 6 aa, synthetic construct
  REGION 1..6: note = synthetic peptide
KTINGS

SEQ ID NO: 38 - protein, 7 aa, synthetic construct
  REGION 1..7: note = synthetic peptide
PGMVWQD

SEQ ID NO: 39 - protein, 6 aa, synthetic construct
  REGION 1..6: note = synthetic peptide
RTQSTG

SEQ ID NO: 40 - protein, 7 aa, synthetic construct
  REGION 1..7: note = synthetic peptide
PGMVWQN

SEQ ID NO: 41 - protein, 6 aa, synthetic construct
  REGION 1..6: note = synthetic peptide
QQNAAP

SEQ ID NO: 42 - protein, 8 aa, synthetic construct
  REGION 1..8: note = synthetic peptide
GQSGAQAA